(19) United States
(12) Patent Application Publication
(10) Pub. No.: US 2019/0358348 A1
(43) Pub. Date: Nov. 28, 2019

(54) CHARACTERIZATION OF BIOLOGICAL TISSUES AT A CELLULAR LEVEL USING RED AND FAR-RED FLUORESCENT DYES

(71) Applicants: IGR-Institut Gustave Roussy, Villejuif Cedex (FR); Mauna Kea Technologies, Paris (FR)

(72) Inventors: Muriel Abbaci, Nogent L'artaud (FR); Odile Casiraghi, Vitry-sur-Seine (FR); Corinne Laplace-Builhe, Montreuil (FR)

(73) Assignees: IGR-Institut Gustave Roussy, Villejuif Cedex (FR); Mauna Kea Technologies, Paris (FR)

(21) Appl. No.: 16/441,634

(22) Filed: Jun. 14, 2019

Related U.S. Application Data
(63) Continuation of application No. 14/401,549, filed on Nov. 17, 2014.
(60) Provisional application No. 61/648,878, filed on May 18, 2012.

(51) Int. Cl. A61K 49/00; A61B 5/00; G01N 21/64

(52) U.S. Cl. CPC A61K 49/003; A61B 5/0071; G01N 21/6428; A61K 49/0021; A61B 5/444; A61K 49/0034; A61B 5/0068; A61B 5/443; G01N 21/6458

(57) ABSTRACT

A method for observing the morphology of a biological tissue is disclosed. The method involves administering a combination of fluorescent dyes into the biological tissue. The combination of fluorescent dyes includes two or more fluorescent dyes selected from a group consisting of: patent blue V, isosulfan blue, toluidine blue, hypericin, indocyanine green, MVAC, and doxorubicin. The method further involves using a microscopic imaging system to form an image of the biological tissue. The microscopic imaging system forms the image based on a contrast resulting from the combination of fluorescent dyes. A concentration of the first fluorescent dye depends on an administration route. A fluorescence of the combination of fluorescence dyes reveals the morphology of the biological tissue at a cellular scale. The fluorescence is observed by means of one of: ex vivo microscopy and in vivo microscopy.

### BACKGROUND

Oral cancer, including that of the lip, tongue, pharynx, and oral cavity, ranks 12th among all forms of cancer. Primary malignancy remains a cancer having a poor prognosis, despite current progress in treatment, due to a generally late diagnosis. White light examination and palpation are generally used to locate the biopsy site. Intraoperative lymphatic mapping, following interstitial injection of radiocolloid and blue dye, have been validated and have become widely accepted as routine surgical procedures, used mainly for breast cancer, cutaneous melanoma, and to a lesser extent head and neck cancer.

Optical diagnosis methods were introduced to improve the differentiation between precancerous and cancerous lesions, and normal tissues, and to detect the sentinel lymph node (Rasmussen J. C. et al., “Lymphatic imaging in humans with near-infrared fluorescence”, Curr Opin Biotechno 2009; 20: 74-822009 and Varghese P. et al., “Methylene Blue Dye—A Safe and Effective Alternative for Sentinel Lymph Node Localization”, The Breast J 2008; 14: 61-7). Clinical studies have been performed using macroscopic fluorescence imaging from endogenously and exogenously induced fluorophores, or using spectroscopic methods such as Raman spectroscopy, fluorescence spectroscopy and elastic scattering spectroscopy. Confocal microscopy was for a long time restricted to fundamental research, but has recently been adapted to the in vivo imaging of suspicious tissues at the cellular level (Kiesslich R. et al., “Confocal laser endoscopy for diagnosing intraepithelial neoplasias and colorectal cancer in vivo”, Gastroenterology 2004; 127: 706-13). Probe-based confocal laser endomicroscopy (pCLE) has become widely used in the past few years, in order to meet the need for high resolution imagery. Such non-invasive approaches, referred to as “optical biopsies”, now make it possible to assess and monitor cancer and other diseases in various organs, using fluorescent dyes.

Hypericin, a hydroxylated phenantroperylenequinone, has been considered for fluorescence diagnosis. The higher uptake of this dye in abnormal, in particular cancerous, cells has already been assessed for its potential to increase the detection rates of bladder cancer (D'Hallewin M-A. et al., “Hypericin-based fluorescence diagnosis of bladder carcinoma”, BJU International 2002; 89: 760-3), and malignant oral lesions (Thong P. S. P. et al., “Clinical application of fluorescence endoscopic imaging using hypericin for the diagnosis of human oral cavity lesions”, Br J Cancer 2009; 101: 1580-4). In the latter study, the diagnostic criterion was based on the red to blue intensity ratio, which is greater in non-malignant than malignant lesions.

Toluidine blue, a well-known metachromatic dye from the Thiazin family, has also been investigated for its potential in wide field examinations of the oral cavity, by staining malignant and premalignant lesions by means of a topical application: according to Epstein, premalignant lesions with a high risk of progression to cancer are preferentially colored using this process. Major false positives results observed during macroscopic examinations with this vital dye are commonly related to inflammatory or traumatic areas. A repeat examination is thus strongly recommended after a two week period, to eliminate these causes (Epstein J. B. et al., 2009). A prior meta-analysis was performed to evaluate the efficacy of toluidine blue in oral cancer screening. It was shown that this approach has a sensitivity ranging between 93.5% and 97.5%, and a specificity ranging between 73.3% and 92.9% in high risk populations.

Methylene blue, a Thiazin dye closely related to Toluidine blue, has been considered for the staining of a superficial layer of the oral and laryngeal epithelium, in order to underline cell structures by contact endoscopy. The nucleus/cytoplasm ratio and nucleus color, size and shape can be analyzed in situ, but despite its potential value as a diagnostic tool, only few studies have described the use of this technique in recent years.

The use of methylene blue's ability to preferentially stain precancerous and cancerous lesions, following topical application in the oral cavity, was proposed by Riaz (Riaz et at., “Methylene blue as an early diagnostic marker for oral precancer and cancer”, SpringerPlus 2013, 2:95). A pilot study on 120 patients showed the sensitivity of this macroscopic method to be 91.4% and the specificity to be 66.6%, with a positive predictive value of 97.7% and a negative predictive value of 33%. False positivity may be attributed to inflamed and irregular lesions. This method was proposed for large-scale oral screening of high risk populations.

Finally, patent blue V, a vital blue dye from the triarylmethane family of dyes, is employed mostly for sentinel lymph node mapping in clinical practice. Patent blue is mainly used in Europe. Its counterpart isomer, isosulfan blue, has been approved by the Food and Drug Administration in the United States. A low concentration of both dyes is sufficient to macroscopically visualize a blue colored lymph node with its afferent lymphatic ducts, following interstitial injection. Macroscopic fluorescence for sentinel lymph node detection has been also described with sodium fluorescein and indocyanine green in various clinical studies (Dan A. G. et al., “1% Lymphazurin vs 10% Fluorescein for Sentinel Node Mapping in Colorectal Tumors”, Arch Surg 2004; 139: 1180-4).

Toluidine Blue, Methylene Blue, Patent Blue, Indocyanine green and Hypericin are dyes that are used today in clinical practice or clinical trials for the macroscopic guidance of diagnostic procedures, with very limited side effects for the patients (Narui K. et al., “Observational study of blue dye-assisted four-node sampling for axillary staging in early breast cancer”, Eur J Surg Oncol 2010; 36: 731-6).

In the present disclosure, we evaluate for the first time the fluorescence properties of the red fluorescent dye Hypericin, and the far-red Toluidine blue (TB), Patent Blue (PB) stains and infra-red indocyanine green (ICG) stains used alone or combined, for the characterization of normal and cancerous head and neck tissue, and the imaging of lymph nodes at the cellular level. High resolution fluorescence imaging of human surgical specimens and rat lymph nodes were achieved using a laptop confocal laser scanning microscope (CLSM), a non linear (multiphoton) microscope, and a Probe-Based Confocal Laser Endomicroscope (pCLE). We then compared the corresponding confocal and non linear images with the results obtained using standard histopathology. A potential multimodal approach combining confocal microscopy, reflectance and fluorescent information, for the improved location of biopsy and sentinel lymph node sites, is also discussed.

### SUMMARY OF THE INVENTION

In at least one aspect, embodiments disclosed herein relate to a method for observing the morphology of a biological tissue, comprising:
using a fluorescent dye on said biological tissue, wherein the fluorescent dye is selected from patent blue V, isosulfan blue, toluidine blue, hypericin, indocyanine green, MVAC, or doxorubicin;
using a microscopic linear or non linear imaging system to form an image of said biological tissue, wherein the fluorescence of said dye reveals the morphology of said tissue at cellular scale.

The applicants show in the present disclosure unknown properties of the above mentioned dyes, either unknown fluorescence properties (ex: patent blue V, isosulfan blue) or unknown ability to reveal contrasted images at cellular scale (ex: hypericin, MVAC, doxorubicin, indocyanine green).

More specifically, the fluorescence properties of patent blue V, Indocyanine green for in situ highlighting of the cellular structure of the sentinel lymph node, and more widely all fluorescent dyes; Hypericin, Toluidine blue, Patent blue V, Indocyanine green, providing morphological information related to malignant head and neck lesions following a topical application, is shown in the present disclosure.

MVAC and Doxorubicin are used for “therapeutic imaging” at the cellular level, allowing the accurate administration and monitoring of the drug biodistribution and a better understanding of the in viva molecular therapeutic efficacy of this approach.

According to an embodiment, the method comprises meeting specific cellular examination conditions by introducing the fluorescent dyes at dyes concentrations which are significantly lower that those traditionally used for current clinical use. Under these new conditions, an acceptable contrast is obtained which allows pathologists to perform a morphological reading of the tissues at cellular scale, this reading allowing the identification of key parameters and cellular structures to support diagnosis.

According to an embodiment, the method comprises introducing patent blue into said biological tissue, wherein the concentration of patent blue is chosen between 0.05 mg/mL (0.005%) and 25 mg/mL (2.5%), depending of the administration route.

According to an embodiment, the method comprises introducing toluidine blue into said biological tissue, wherein the concentration of toluidine blue is chosen between 0.1 mg/mL (0.01%) and 20 mg/mL (2%).

According to an embodiment, the method comprises introducing hypericin into said biological tissue, wherein the concentration of hypericin is chosen between 0.252 μg/mL (0.5 μM) and 5.02 μg/mL (10 μM).

According to an embodiment, the method comprises introducing indocyanine green into said biological tissue, wherein the concentration of indocyanine green is chosen between 0.05 mg/ml and 5 mg/ml, depending of the administration route.

According to an embodiment, the fluorescence is observed by means of ex vivo linear or non linear microscopy.

According to an embodiment, the fluorescence is observed by means of in vivo linear or non linear microscopy.

According to different embodiments, introducing of the fluorescent dye is made either intravenously, subcutaneously, or by submucosal or topical administration.

According to different embodiments, the biological tissue is selected from abdomino-pelvic cavity, thoracic cavity, head and neck sphere, lymphatic system.

According to an embodiment, microscopy is performed by means of fiber based endomicroscopy.

The following table summarizes in a non limitative way the possible use of these dyes in endomicroscopy:

### BRIEF DESCRIPTION OF THE DRAWINGS

FIG. 1: Tip of the flexible miniprobe gastroflex UHD, with a typical sample for the study (A), a 240 μm field of view image from a tumor sample produced from one snapshot taken with the Cellvizio (B), and a reconstructed image made with the video mosaicing technique, to provide a more complete representation of the region of interest (C).

FIG. 2: Representative fluorescence imaging of tumoral and non-tumoral tissue after Toluidine Blue staining, and corresponding histological images for each case. High-grade dysplasia from the ventricular band, observed with CLSM (A), pCLE (B), and HES (C). In situ papillary carcinoma imaged with CLSM (D), showing nuclear details (white arrow) and a mitosis figure (yellow arrow), which are discernible in several cells, pCLE (E) and HES (F). Well-differentiated HNSCC imaged with CLSM (G), pCLE (H) and HES (I). Note the stromal fibroblasts (yellow star) surrounding the tumor cells.

FIG. 3: Representative fluorescence imaging of tumoral and non-tumoral tissue after MB staining, and corresponding histopathologic images for each case. Squamous epithelium from the lateral border of the tongue, analyzed with CLSM (A), pCLE (B) and HES (C); poorly differentiated non-keratinizing HNSCC from the larynx observed with CLSM (D), with inflammatory cells characterized by small round nuclei (white ellipse), pCLE (E) and HES (F). Finally, invasive, moderately differentiated, non-keratinizing HNSCC invading the muscle, observed with CLSM (G), pCLE (H) and HES (I). Note the morphology of the typical normal muscle cells in fluorescence mode (white arrows), as compared to the corresponding morphology revealed by HES.

FIG. 4: Representative fluorescence imaging of tumoral and non-tumoral tissue after PB staining, and corresponding histopathological images for each case. Squamous epithelium with CLSM (A), pCLE (B) and HES (C); Partial squamous metaplasia of the epithelium of an accessory salivary glandular canal posterior wall of the pharynx viewed with CLSM (D), pCLE (E) and HES (F); poorly differentiated HNSCC viewed with CLSM (G), pCLE (H) and HES (I).

FIG. 5: Representative fluorescence imaging of a tumor after hypericin staining, and corresponding histopathological images: moderately differentiated squamous cell carcinoma observed with CLSM (A), pCLE (B) and HES (C).

FIG. 6: Representative fluorescence imaging of a larynx HNSSC after Indocyanine green staining. Ex vivo, squamous cell carcinoma stained by topical application of a 0.25 mg/ml indocyanine green solution for 10 s. The sample was then imaged with a linear confocal microscope CLSM (λex=635 nm) (A) and a fibered confocal microscope (λex=785 nm) (B).

FIG. 7: Multiparametric fluorescence mode imaging of tumor specimens acquired by CLSM. (A) HNSCC after TB staining (green and inset). The carcinoma trabeculae appear to be green and are associated with a major fibrous stroma (blue) detected by autofluorescence (λex=405 nm and λem=420-500 nm), (B) a well differentiated HNSCC after MB staining (green and inset) with a collagenous and elastin stromal network (blue) detected by autofluorescence (λex=405 nm and λem=420-500 nm). The yellow arrows indicate keratin pearls. (C) A verrucous squamous cell carcinoma was discerned after TB staining (green signal and inset), but the parakeratosis cell nuclei (white ellipse) could be detected only after ACF staining (red signal). (D) A well-differentiated HNSCC with some cell borders (white arrow) perceived after MB staining (green signal and inset). However, there is double staining with ACF (red signal) highlighted nuclei, in an area where the MB was not loaded (yellow arrow).

FIG. 8: Multiparametric imaging of a tumor specimen in fluorescence and reflectance mode acquired with a CLSM, using a 40× objective. (A) poorly to moderately differentiated HNSCC after MB staining; (B) autofluorescence signal (in blue): collagenous and elastin fibers can be seen more clearly (λex=405 nm and λemi=420-500 nm); (C) reflectance signal (in red, λ=488 nm) where stroma cells and carcinoma cells are present, and (D) an overlay of the green, red and blue signals from the sample. Yellow areas indicate colocalization of the MB and reflectance signal, and pink areas indicate colocalization of the autofluorescence and reflectance signals. Macrophages were also visible (yellow arrow). Scale bar 50 μm

FIG. 9: Representative fluorescence imaging of a non-pathological rat lymph node after subcutaneous injection of blue dyes: typical TB staining observed with CLSM (A), pCLE (B) and corresponding HES (C); typical MB staining with CLSM (D), pCLE (E) and corresponding HES (F); typical PB staining with CLSM (G), pCLE (H) and corresponding HES (I). Yellow arrows indicate lymphoid follicles, green arrows interfollicular zones, red arrow the sinus with physiological sinusal histiocytosis, and blue arrows adipocytes around the node.

FIG. 10: Non linear imaging of lymph node stained with Indocyanine green. Ex vivo, lymph node stained by topical application of a 0.25 mg/ml indocyanine green solution for 10 s.

The sample was then imaged with a multiphoton microscope at 830 nm.

The node capsule on which some adipocyte layers are attached can be seen on the left side of the frame and the inner cortical region of the ganglion on the right side.

We note that in the cortical area, the fluorescence of ICG is perinuclear making cell nuclei came out negative, but the fluorescence is also located in the pericellular space.

### ABBREVIATIONS

CLSM: Confocal Laser Scanning Microscopy
pCLE: Probe-based Confocal Laser Endosmicroscope
HES: histological slide stained with Haematoxylin and Eosin-Safran
HNSSC: Head and Neck Squamous Cell Carcinoma

### DETAILED DESCRIPTION

In the present disclosure, all percentage concentrations refer to weight/volume (w/v) percent, except otherwise indicated. The solutions are aqueous solutions, except otherwise indicated.

### Materials and Methods

Materials

Hypericin was purchased from Invitrogen (Cergy Pontoise, France), MB from Aguettant laboratory (MB 1% injection, Lyon, France), Toluidine Blue (TB) from Fluka (Sigma Aldrich, St Quentin Fallavier, France), Patent Blue (PB) from Guerbet (sodium patent blue V 2.5% injection, Villepinte, France), Acriflavine hydrochloride (ACF) from Sigma Aldrich (St Quentin Fallavier, France), Infracyanine 25 mg (ICG) from SERB Laboratoires, Paris, France) and acetic acid was provided by Gyneas laboratories (acetic acid 5%, Goussainville, France).

Human Specimen

A total of 25 patients, who underwent total (pharyngo-) laryngectomy or oral and maxillofacial surgery for squamous cell carcinoma (HNSCC) at the Institut Gustave Roussy (Villejuif, France), were included in the prospective protocol (from January 2009 to April 2011). The Institutional Review Board approved the study, and informed consent was obtained from all patients. 30 freshly collected tissues with a maximum surface area of 1 cm³ were clinically distinguished as non-tumoral mucosa and tumor tissue by the pathologist before fluorescence imaging.

Animal Lymph Nodes

10 Male Wistar rats weighing 350 g were purchased from Janvier. Animal care and studies were performed according to the European convention for the protection of Vertebrate

Animals used for Experimental and other Scientific Purposes, EU directives and the French Law on Statute on Animal Experiments. All experimental protocols were approved by the Committee for Care and use of Animals in Experiments at the Institut Gustave Roussy. The rats were maintained in standard cages in isolators, were housed with a 12-hour light/dark cycle at 22-24° C. and 50% humidity, and were administered with ad libitum food diets and water.

Preparation of the Human Surgical Specimens

General Sample Pretreatment

Head and neck mucosa are often keratinized. Since these layers of keratin are barriers for dye and light diffusion into the tissue, they may be reduced at the luminal surface of the sample by tape-stripping (3M surgical tape).

Hypericin Staining

Fresh surgical samples were treated with 10% acetylcystein for 2 min and incubated with a fresh 8 μM Hypericin solution for 30 min at 37° C.

Toluidine Blue Staining

Fresh samples were rinsed with 1% acetic acid, and briefly stained with TB 1% (w/v). After a waiting period (up to 5 min) needed to optimize fluorescent dye diffusion into the tissue, the samples were rinsed in 1% acetic acid.

Methylene Blue Staining

The surgical specimens were treated with 10% acetylcysteine for 2 min and briefly loaded with 0.25% MB. After a waiting period (up to 5 min), the samples were rinsed in a 0.9% sodium chloride solution.

Patent Blue Staining

The surgical specimens were treated with 10% acetylcystein for 2 min, then immerged in a 0.025% patent blue solution for 10 s, and finally rinsed in a 0.9% sodium chloride solution.

Indocyanine Green Staining

The surgical specimens were treated with 10% acetylcystein for 2 min, then topically stained with a 0.25 mg/ml ICG solution for 10 s, and finally rinsed in a 0.9% sodium chloride solution.

Multiple Staining of the Tissue

In a small number of cases, the samples were additionally stained with 0.01% (w/v) acriflavine hydrochloride for 45 s, and then rinsed in a 0.9% sodium chloride solution for 1 min, to enable the discussion of a potential combination of Toluidine Blue-Acriflavine and Methylene Blue-Acriflavine, for the purposes of deriving additional morphological information.

Lymph Node Preparation

The animals were briefly anesthetized by inhalation of isoflurane (2.5% for induction, 1.5% for maintenance). Then, 100 μL of either 1.25% Patent blue, 1% toluidine blue, or 0.25% methylene blue were injected subcutaneously into the footpad of the animals' hindpaw, and allowed to diffuse towards the lymph node for 5 min. The rats were euthanized by means of an intravenous pentobarbital injection and the popliteal/and or inguinal lymph nodes which had a macroscopically blue appearance were dissected. The ex vivo lymph nodes were then imaged by confocal laser scanning microscopy and probe-based confocal laser endomicroscopy (pCLE).

Confocal Laser Scanning Microscopy (CLSM)

We performed fluorescence imaging using a DMI400 inverted microscope (Leica TCS SPE, Mannheim, Germany). The head and neck tissue and lymph node were placed in a 35 mm μ-dish (Ibidi, Biovalley, France). The morphology and architecture of the tissues were assessed using the following laser lines: Acriflavine hydrochloride (excitation wavelength λₑₓ=488 nm; emission wavelength λₑₘ=500-600 nm); Hypericin (excitation wavelength λₑₓ=532 nm; emission wavelength λₑₘ=550-650 nm); TB, MB and PB (λₑₓ=635 nm; λₑₘ=650-800 nm). The images were formed using either a 10× objective lens (dry/NA 0.3) corresponding to a 1.1 mm×1.1 mm field of view, or a 20× objective lens (dry NA 0.7) with a 550 μm×550 μm field of view, or a 40× objective lens (oil/NA 1.25) with a 275 μm×275 μm field of view. Z series were produced with 5 μm steps, in order to image the cellular distribution and morphologies of different tissue layers.

Non Linear Laser Scanning Microscopy (Multiphoton)

We performed fluorescence imaging using a SP8 inverted multiphoton microscope (Leica TCS SPE, Mannheim, Germany). The head and neck tissue and lymph node were placed in a 35 mm μ-dish (Ibidi, Biovalley, France). The morphology and architecture of the tissues were assessed using 830 nm laser line, pulse width 100 fs at 830 nm and repetition rate of 80 Mhz. Fluorescence was collected through a band pass filter 580-680 nm. The images were acquired using a 20× objective lens (dry/NA 0.75) corresponding to a 550 μm×550 μm field of view. Z series were produced with 5 μm steps, in order to image the cellular distribution and morphologies of different tissue layers.

Probe-Based Confocal Laser Endomicroscope (pCLE)

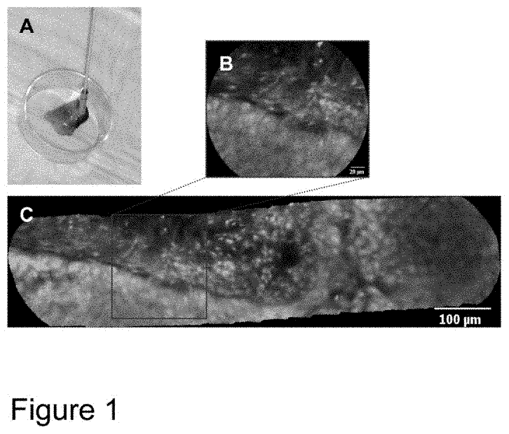

As CLSM and multiphoton microscope are benchtop instruments unsuitable for in vivo exploration, fluorescence images were then additionally recorded using a fibered imaging system, Cellvizio®, kindly provided by Mauna Kea Technologies (Paris, France), which has been successfully tested for clinical studies of gastrointestinal, urinary and pulmonary tracts. The pCLE comprises a flexible, fibered miniprobe connected to a laser unit, which is equipped with a laser diode operating at 660 nm (for TB, MB and PB excitation) or 568 nm (for Hypericin excitation), a rapid scanning laser (frame rate from 8 to 12 images per second), and a photodiode to detect the fluorescence signal. Conversely to conventional histopathology, the “optical slices” obtained from the specimens are not transversal but en face to the tissue surface sections. The data was acquired using a confocal miniprobe gastroflex UHD (Mauna Kea technologies, Paris, France), which has a 240 μm diameter field of view. This fiber bundle, composed of 30,000 optical fibers, has a transverse resolution of 1 μm and an axial resolution of 10 μm. The images are taken at depths varying between 55 μm and 65 μm below the surface of the skin. Image reconstruction was performed using video mosaicing software, in order to achieve a more complete representation of a given region of interest through the generation of a larger static image (FIG. 1). Tissue auto fluorescence control images were also recorded, on carcinoma and non tumoral mucosa from various anatomical sites.

Histopathology

The samples previously analyzed with pCLE and CLSM were fixed either in formol for the case of head and neck tissue, or in Finefix for the lymph node, then embedded in paraffin, sectioned at 3 μm thicknesses perpendicularly to the surface mucosa, and stained with Haematoxylin and Eosin-Safran (HES). The histological examinations and fluorescence images from the head and neck tissues were read and interpreted by a pathologist specialized in head and neck pathology.

### Results

Head and Neck Tissue Imaging with Toluidine Blue

Efficient fluorescence imaging of the specimens was straightforward after TB staining. Optimal diffusion 60 μm below the surface (focal plane of gastroflex UHD) was observed 15 min after dye loading.

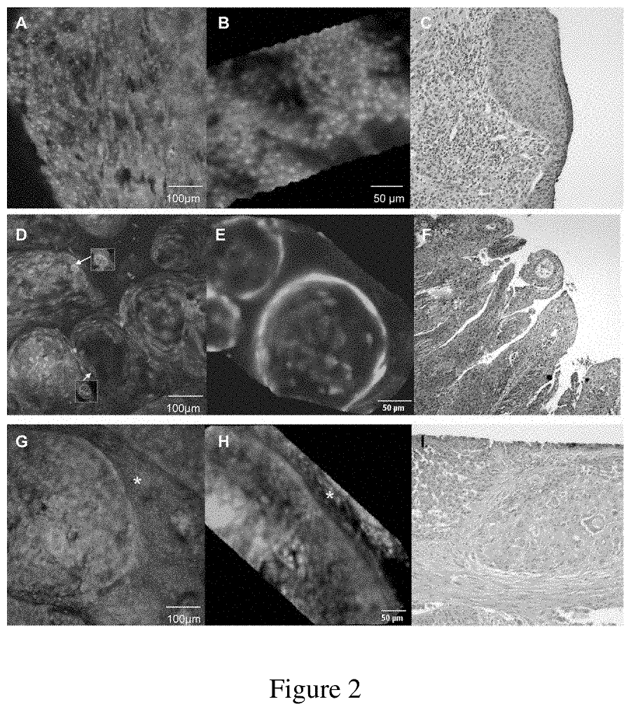

Bright fluorescence in the nuclei and diffuse staining within the cytoplasm of the cells were observed with both systems, in dysplasia from metaplasic squamous epithelium ventricular band specimens (FIGS. 2a and 2b). A slight heterogeneity in size and shape of the nuclei could be observed, and was associated with irregular cell distributions. The images were collected only at depths in the range from 0 to 65 μm below the epithelium surface, due to the limited extent of dye diffusion, and the pCLE focal plane which is restricted to the range between 55 μm and 65 μm. The high grade of the dysplasia was confirmed by the pathologist by means of HES slide examination. On highly keratinized samples, some deeper areas were not visible, probably as a consequence of restricted dye diffusion, due to residual keratin at the mucosal surface and the resulting increase in epithelium thickness.

An in situ papillary carcinoma is shown in FIGS. 2d and 2e. A typical microscopic papillary architecture was seen with both systems. An abnormal cell distribution and atypia were discernible. Papillary structures appeared en face to be circular, since they were imaged perpendicularly to their axis. The intense circular staining at the cells' periphery could possibly be attributed to a thin layer of keratin at the surface of the papillary structures. Interestingly, chromatin (FIG. 2d, white arrow) as well as mitosis could be discerned in the nuclei of some tumoral cells (FIG. 2d, yellow arrow) using CLSM.

The images of well-differentiated HNSCC reveal a heterogeneous cellular distribution with a high cellular density, surrounded by oriented stromal cells (yellow star) (FIGS. 2g and 2h). An undifferentiated nucleus and cytoplasm staining were noticed in the case of cancerous cells, due to their high nucleus to cytoplasm ratio. The surrounding oriented cells (fibroblasts and myofibroblasts) in stroma could be distinguished from cancerous cells (FIGS. 2d and 2e).). The capillary network could not be identified by topical application of Toluidine blue. All TB images produced by both confocal imaging systems were interpretable by the pathologist. Thanks to the capability of CLSM to produce a series of images at different depths, the cellular and sub-cellular details could be more easily discriminated in superficial planes. This is a consequence of the TB fluorescence gradient present between the surface and the depth of the tissue.

Head and Neck Tissue Imaging with Methylene Blue

The specimens were imaged immediately after MB staining. We observed a progressive decrease in fluorescence intensity and contrast imaging, over a period of 2 hours, due to dye leakage from the tissue.

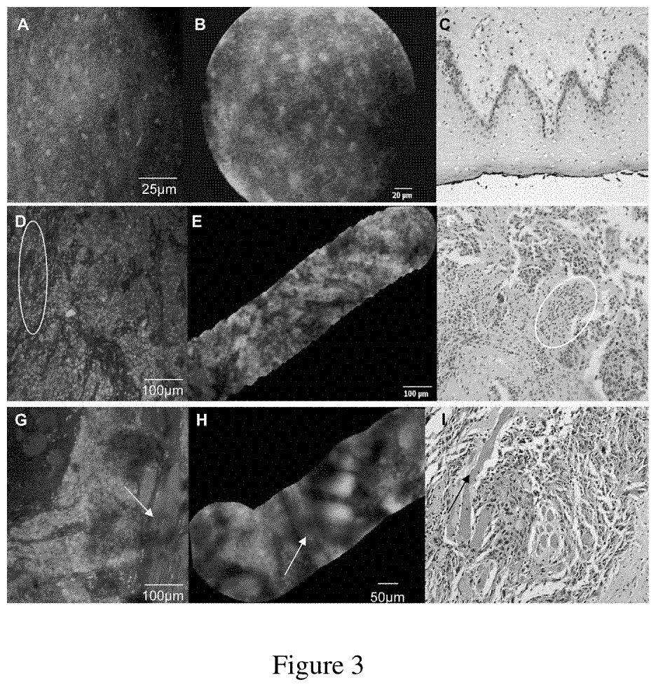

On non-tumoral samples, such as squamous epithelium from the lateral border of the tongue (FIGS. 3a and 3b), we observed diffuse staining in the cell cytoplasm and a higher fluorescence intensity in the nuclei, with both imaging systems. As could be expected, a regular nuclear distribution can be clearly seen, and the cells with a low nucleus to cytoplasm ratio were confirmed with superficial layer analyses based on conventional histology (FIG. 3c).

On HNSCC specimens, cancerous cells, heterogeneously arranged in trabeculae (FIGS. 3d and 3e, here a moderately-differentiated HNSCC) or lobules, were easily discerned from stroma after MB loading. An increase in cellular density could also be noticed. Cytological atypia were recognized, although we regret the lack of contrast achieved with pCLE. With CLSM, it was also possible to distinguish inflammatory cells with small round nuclei (FIG. 3d, white ellipse) from fibroblasts exhibiting spindle nuclei. Moreover, on a surgical specimen from invasive carcinoma, we were able to clearly observe high density cancerous cells infiltrating the muscles (white arrows) with both systems (FIGS. 3g and 3h).

Head and Neck Tissue Imaging with Patent Blue

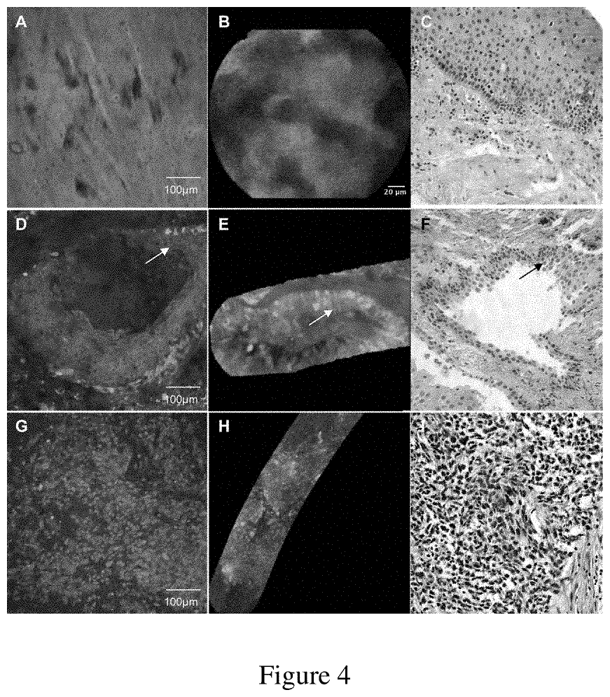

The specimens were imaged immediately after PB staining. A rapid decline (over a period of 1 to 2 hours) in the quality of contrast imaging was observed due to blue dye leakage. FIG. 4 presents high quality fluorescence images of squamous epithelium (4a, 4b), metaplasia ducts (4d, 4e), and HNSCC (4g, 4h), and these can be compared with the corresponding conventional histopathological images (4c, 4f and 4i). Images of non pathological tissue show a PB dye distribution throughout the cell cytoplasm. On cancerous samples, heterogeneous cell morphology was easily discernable, and was associated with multiple trabeculae with an intense staining of nuclei. An increased cellular density was also readily noticeable.

Head and Neck Tissue Imaging with Hypericin

Samples were imaged after 30 min of immersion in a hypericin solution at 37° C. Cytoplasmic and nuclear membranes could be discerned, and were associated with diffuse fluorophore staining in the cytoplasm. A heterogeneous cell distribution could be observed under fluorescence in the case of tumor tissues, such as in moderately differentiated squamous cell carcinoma (FIG. 5).

Head and Neck Tissue Imaging with Indocyanine Green

The specimens were imaged immediately after ICG staining. A slight decrease of fluorescence was seen due to ICG beaching under excitation light, but no dye leakage was observed over the imaging procedure. FIG. 10 shows high quality fluorescence images of HNSCC with the fluorescence of ICG distributed throughout the cell cytoplasm. For both CLSM (A) and pCLE (B) images, the cell morphology was easily discernable and the disruption of the tissue architecture was well visible on this highly differentiated carcinoma,

Multipectral and Multimodal Confocal Imaging of Head and Neck Tissue

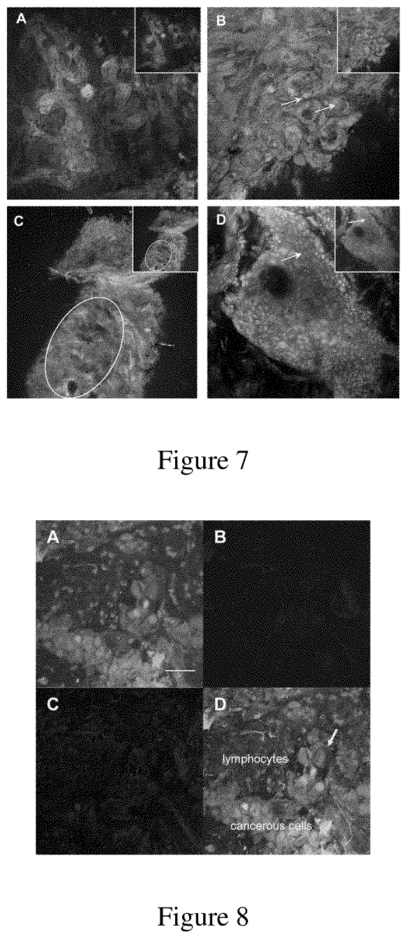

We assessed the potential of a multiparametric analysis to improve the image quality of HNSCC specimens, since the full detailed structure of a tissue cannot be stained by only one of the above-mentioned dyes. Initially, the autofluorescence of tissue specimens, following laser excitation at 405 nm, was combined with TB or MB staining. A HNSCC sample was firstly loaded with TB, highlighting irregular trabeculae bordered by weakly fluorescent tissue (FIG. 7a). When the autofluorescence signal was recorded additionally, the pathologist could efficiently recognize a major fibrosis, which was correlated with the corresponding H&E section. In a second case, carcinoma lobules of well-differentiated HNSCC stained with MB could easily be recognized. Stroma characterization was supplemented by the image of the elastin-rich matrix based on the autofluorescence signal (FIG. 7b). In this sample, keratin pearls were also stained with MB (yellow arrows). In all images, the additional information provided by autofluorescence made it possible to come closer to a conventional representation of tumor tissue by H&E section.

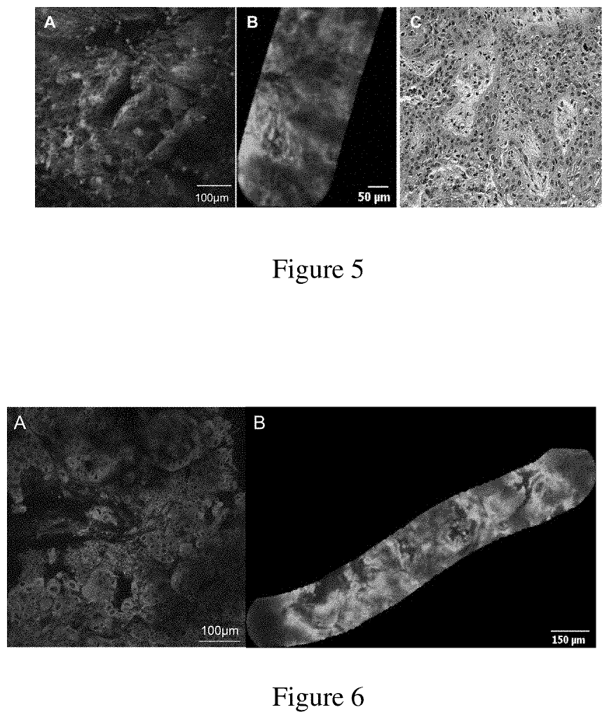

In a second approach, surgical specimens were dually stained with either Toluidine blue-Acriflavine or Methylene blue-Acriflavine. Acriflavine (ACF) is a green fluorescent dye that intercalates into the DNA bases, and strongly stains the cell nuclei. FIG. 6c shows a verrucous squamous cell carcinoma after Toluidine blue-Acriflavine loading, with a progressive differentiation between cancerous cells and small cells in a disorganized architecture, to flattened cells with major keratinisation and a trend to whirlpool. The keratosis process was clearly visible with both single fluorescent dyes, but parakeratosis was revealed only by ACF, since the nuclei and keratin could not be distinguished in this zone with TB alone (white ellipse). FIG. 7d presents an example of a well-differentiated HNSCC stained with ACF and MB. Besides nuclei and cytoplasm staining, some cell borders (white arrow) were discerned with MB. ACF and MB co-localization was observed on most parts of the tumor tissue. However, we noticed anon MB-loaded area in which some nuclei became visible with ACF. Finally, a well-differentiated HNSCC loaded with MB was also imaged using autofluorescence and reflectance as additional parameters, in order to refine the pathologist's interpretation. Reflectance imaging makes use of backscattered light from the tissue, with various refractive indices linked to its structure. The resulting multi-color image provided additional data from the disturbed architecture; the MB image exhibits discernible tumor cells and inflammatory cells (FIG. 8a). In FIG. 8d, the inflammatory cells were composed of lymphocytes seen as small cells with round nuclei, and macrophages in the form of large cells with various shapes, containing a small nucleus (yellow arrow).

Microscopic Rat Lymph Node Imaging

Only healthy lymph nodes were imaged in our feasibility study.

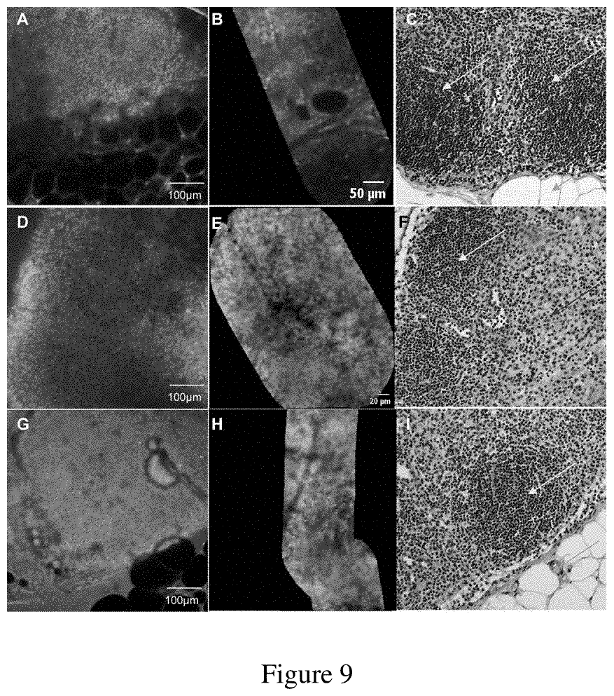

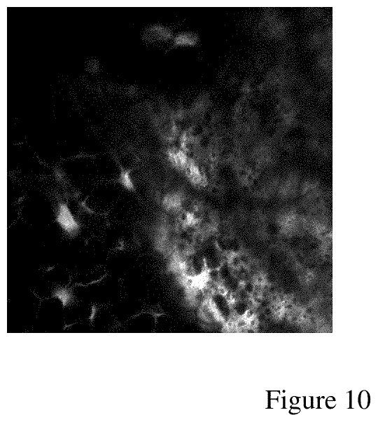

Following sub-cutaneous injection of PH, TB or MB, the diffusion of the blue dyes to the popliteal and/or inguinal lymph node was macroscopically visible, after a period of only 5 min. The lymph nodes were imaged ex vivo using CLSM and pCLE (FIGS. 9a, d, g and 9b, e, h). The lymphocytes were uniformly stained with MB, TB and PB. A homogeneous distribution of lymphatic cells could easily be distinguished under the node capsule.

In multiphoton microscopy of ICG stained samples, a localized staining was seen in the cytoplasm of the cell in the cortical area of the modes. The cell nuclei appeared black on the images that signs an absence of nuclear fluorescence, after a topic administration of ICG dye. A quite homogeneous cell distribution could be observed as the sample was a non pathologic and non inflammatory lymph node. (FIG. 10).

The fluorescence properties of patent blue V, and ICG for in situ highlighting of the cellular structure of the sentinel lymph node have never been previously described. This new approach could provide morphological information related to malignant lesions after only a topical application of the dye on the tissue.

### Discussion

In the present disclosure, we propose a new approach to fluorescence imaging at the cellular level, more particularly for the imaging of the head and neck tissue and for lymph node examinations, especially that of the sentinel lymph node.

Fluorescence features of TB, MB, PB, ICG and Hypercin are used to produce images of high quality at the cell and tissue level, to enable the pathologists to make a diagnosis on the basis of these “optical biopsies”. The information on the tissue's morphology and architecture provided by these dyes is assessed on human tumoral and non-tumoral head and neck specimens, and on animal lymph nodes.

In clinical practice, precancerous and cancerous lesions present a preferential chromoscopic dye uptake for TB and MB at the macroscopic level (Epstein J. B. et al., 2009). A less expected result is that a significant fluorescence signal can also be found in the squamous epithelium, for both dyes in the present disclosure. On HNSCC specimens, tumor cells with their surrounding stroma are microscopically well discerned with both dyes, from the homogeneous and regular staining of squamous epithelium. Cancerous lesions can be precisely distinguished at the cellular level, via cytological abnormalities such as changes in nuclear size and shape (anisonucleosis), an increased nucleus/cytoplasm ratio, and even at the tissue level via clustering of the cells and irregular cell architecture. The confocal CLSM images also reveal mitosis patterns with a level of detail similar to that obtained with conventional biopsies. Interestingly, topical administration of these red and far red fluorescent contrast agents also provides helpful staining of keratinization anomalies, such as keratin pearls.

Contrary to MB and TB, which are mostly used topically, PB is already routinely used in clinical routine, via interstitial administration (mostly peritumoral injection), at concentrations allowing fluorescence imaging to be applied at the microscopic scale. The topical delivery of these dyes on tissues is also relevant to get images that can be interpretable by pathologists. When using a topical delivery mode, the limited diffusion of the dyes into the tissue must be taken into account, as it will limit the imaging depth to approximately 60 μm below the surface, as observed with MB and TB. Further, we may proceed with mechanical stripping of the surface mucosa to remove most of the keratin layers in HNSCC specimen and then improve the production of readable images at the microscopic scale. Tissue labeling with both blue dyes (TB, PB) occurs almost immediately (in less than one minute) and their clearance on ex vivo specimens can be compatible with the timing of surgical procedures (more than 15 min). When compared to the blue dyes and ICG, cellular imaging performed with hypericin requires a longer time interval after staining, in order to provide readable microscopic images (at least 30 min). This duration is however compatible with conventional clinical protocol. Macroscopic false positives with MB, TB and hypericin following topical application are usually associated with an inflammation or regenerative tissues. All of the confocal microscopy or endomicroscopy images produced with these red and far-red fluorescent dyes were of sufficient quality to be interpreted by the pathologists. However, these fluorescent markers taken alone may have some limitations for the identification of tumor stromal disorders, because the fibrosis network may have an only slightly fluorescent appearance. We showed that multi-modal microscopic imaging can contribute to gain additional useful information in the images, allowing improved identification of the fiber network and inflammatory cells. The potential use of reflectance by confocal microscopy was successfully investigated to diagnose laryngeal lesions and for the detection of in vivo oral lesions. Although unstained tissue can be imaged in reflectance mode, the induced fluorescence mode permits improved structural imaging with a high signal to noise ratio, and reveals features closer to those observed by histopathology.

In the present disclosure, we also show that, by combining images from the fluorescence induced by far-red contrast agents, together with autofluorescence or reflectance signals, all cellular and extracellular structures can be identified with an accuracy close to that achieved with conventional histology. The multi-spectral imaging described in the present disclosure, combining the use of dual fluorescent staining (ACF dye and near-infrared fluorescent dyes), indeed provides supplementary histological data which improves the pathologist's diagnostic capabilities.

PB is used routinely to detect sentinel lymph nodes, which become macroscopically blue after blue dye injection (Gill J. et al., “Sentinel Lymph Node Biopsy in Breast Cancer: An Analysis of the Maximum Number of Nodes Requiring Excision”, The Breast J 2011; 17: 34). Clinical studies have also described the interstitial injection of MB for sentinel lymph node localization (Varghese P. et al., “Methylene Blue Dye—A Safe and Effective Alternative for Sentinel Lymph Node Localization”, The Breast J 2008; 14: 61-7). However, animal studies showed results based on the macroscopic red fluorescence signal from the lymph node after MB injection. In the present disclosure, we have for the first time tested, in healthy rats, the potential of associating chromoscopic lymph node detection with fluorescence imaging at the microscopic level, following the injection of blue dyes and Indocyanine green. The lymph node's normal histology was discerned by the pathologist, and this result highlights the potential of pCLE for the in situ imaging of lymph nodes, prior to sentinel lymph node resection. We also showed unexpected outstanding fluorescence of ICG using non linear excitation. This result allows for a good description of the cortical cell structure in rat lymph nodes.

All of the contrast agents used in the present disclosure are approved for use in humans, and for their morphological staining capability. The new imaging technique proposed here involves no changes in clinical protocols that have already been approved by health authorities, using the blue dyes for sentinel lymph node protocol in breast cancer research or in the melanoma, hypericin for photodynamic therapy, ICG for retinal angiography.

It is noteworthy that the concentrations of the markers we propose to use are either equal to the maximum clinical dose administered to patients, and more often 5 to 10 times lower than the clinical doses approved.

The administration routes of fluorescent markers that we propose to use are identical to those already used in clinical routine for which very little or no evidence of deleterious effects for the patients have been observed after several years of clinical practice (see Uhara H et al. “Applicability of radiocolloids, blue dyes and fluorescent indocyanine green to sentinel node biopsy in melanoma”, J. Dermatol. 2012 Sep. 20. doi: 10.1111/j.1346-8138.2011.01340.x.). Moreover, we demonstrate that the major route can be a localized topical application, for most of the tissues to be imaged.

These new approaches of functional or molecular imaging, which improve the early diagnosis and development of safer and more effective therapeutics, have been shown in confocal endomicroscopy. In the same context, therapeutic molecules having intrinsically fluorescent properties can also be considered, such as doxorubicin or MVAC, for “therapeutic imaging” at the cellular level, allowing the accurate administration and monitoring of the drug biodistribution and a better understanding of the in vivo molecular therapeutic efficacy of this approach.

When combined with fibered microscopic imaging, the dyes assessed in this study provide considerable assistance to the surgeon, through improved clarification of the content of zones which are not well understood when perceived at the mesoscopic level. pCLE can be performed in conjunction with a visual investigation, following topical application of the dyes. The physician can determine the nature of a suspicious zone at the time of the first examination, such that repeat examinations to check for inflammation and traumatic lesions would be unnecessary. Moreover, since pCLE is suitable for real-time in vivo histopathological examinations, this imaging technique can assist the clinician by revealing the need to perform a biopsy, or allowing the extent of a surgical resection to be determined. Macroscopic and microscopic fluorescent examinations are complementary for the diagnosis of tissue anomalies, and can provide physicians a fast, new, non-invasive, multiscale approach to medical imaging.

1.-17. (canceled)

18. A method for observing a morphology of a biological tissue, the method comprising:
administering a fluorescent dye into said tissue, wherein the fluorescent dye is one selected from the group consisting of patent blue V, isosulfan blue, toluidine blue, hypericin, indocyanine green, MVAC, and doxorubicin; and
using a microscopic imaging system to form an image of said biological tissue, wherein the fluorescence of said fluorescent dye reveals the morphology of said tissue at cellular scale,
wherein the fluorescence is observed by means of in vivo linear or non-linear microscopy in real-time, and
wherein the concentration of the fluorescent dye depends on an administration route.

19. The method according to claim 18, wherein the fluorescent dye is indocyanine green.

20. The method according to claim 18, wherein the fluorescent dye is patent blue.

21. The method according to claim 20, wherein a concentration of patent blue is chosen between 0.005% and 2.5%.

22. method according to claim 18, wherein the fluorescent dye is toluidine blue.

23. The method according to claim 22, wherein a concentration of toluidine blue is chosen between 1% and 2%.

24. The method according to claim 18, wherein the fluorescent dye is hypericin.

25. The method according to claim 24, wherein a concentration of hypericin is chosen between 0.5 μM and 10 μM.

26. The method according to claim 18, wherein microscopy is performed by fiber based endomicroscopy.

27. The method according to claim 26, wherein the microscopy imaging process is confocal.

28. The method according to claim 27, further comprising observing cellular and extracellular structures by combining images from the fluorescence induced by far-red contrast agents together with autofluorescence or reflectance signals.

29. The method according to claim 18, wherein the administration of the fluorescent dye is subcutaneous.

30. The method according to claim 18, wherein the administration of the fluorescent dye is submucosal.

31. The method according to claim 18, wherein the administration of the fluorescent dye is topical.
